(19) United States
(12) Patent Application Publication
(10) Pub. No.: US 2026/0094666 A1
(43) Pub. Date: Apr. 2, 2026

(54) FOLD CONDITIONED PROTEIN STRUCTURE GENERATION

(71) Applicant: NVIDIA Corporation, Santa Clara, CA (US)

(72) Inventors: Karsten Kreis, Vancouver (CA); Tomas Geffner, Santa Clara, CA (US); Kieran Didi, Euskirchen (DE); Zuobai Zhang, Santa Clara, CA (US); Arash Vahdat, San Mateo, CA (US); Danny Reidenbach, San Jose, CA (US); Zhonglin Cao, San Jose, CA (US); Emine Kucukbenli, Melrose, MA (US); Mario Geiger, Lausanne (CH); Chris Dallago, Munich (DE)

(21) Appl. No.: 19/234,113

(22) Filed: Jun. 10, 2025

Related U.S. Application Data
(60) Provisional application No. 63/702,066, filed on Oct. 1, 2024.

(51) Int. Cl. G16B 15/20; G06N 3/0475; G16B 40/20; G16B 40/30

(52) U.S. Cl. CPC G16B 15/20; G06N 3/0475; G16B 40/20; G16B 40/30

(57) ABSTRACT

De novo protein design, the rational design of new proteins from scratch with specific functions and properties, is a grand challenge in molecular biology. Recently, deep generative models have emerged as a novel data-driven tool for protein engineering. However, current diffusion- and flow-based models generally synthesize backbones only, without sequence or side chains, while protein language models often model sequences instead. The present disclosure provides flow-based protein structure generation which can be conditioned on a given fold class.

### CLAIM OF PRIORITY

This application claims the benefit of U.S. Provisional Application No. 63/702,066 titled “SCALING FLOW-BASED PROTEIN STRUCTURE GENERATIVE MODELS,” filed Oct. 1, 2024, the entire contents of which is incorporated herein by reference.

### TECHNICAL FIELD

The present disclosure relates to protein structure synthesis.

### BACKGROUND

De novo protein design, the rational design of new proteins from scratch with specific functions and properties, is a grand challenge in molecular biology. Recently, deep generative models have emerged as a novel data-driven tool for protein engineering. Since a protein's function is mediated through its structure, a popular approach is to directly model the distribution of three-dimensional protein structures, typically with diffusion- or flow-based methods. Such protein structure generators usually synthesize backbones only, without sequence or side chains, in contrast to protein language models, which often model sequences instead, and sequence-to-structure folding models like AlphaFold.

Previous unconditional protein structure generative models have only been trained on small datasets, consisting of no more than half a million structures at maximum. Moreover, their neural networks do not offer any control during synthesis and are usually small, compared to modern generative artificial intelligence (AI) systems in domains such as natural language, image or video generation. There, we have witnessed major breakthroughs thanks to scalable neural network architectures, large training datasets, and fine semantic control.

There is thus a need for addressing these issues and/or other issues associated with the prior art. For example, there is a need to provide flow-based protein structure generation which can be conditioned on a given fold class.

### SUMMARY

A method, computer readable medium, and system are disclosed for protein structure synthesis. A synthetic protein structure of one or more fold classes is generated by a flow-based generative model conditioned on an input fold class parameter indicating the one or more fold classes. The synthetic protein structure is output.

### BRIEF DESCRIPTION OF THE DRAWINGS

FIG. 1 illustrates a flowchart of a method for protein structure synthesis, in accordance with an embodiment.

FIG. 2 illustrates a flow-based generative model architecture for protein structure synthesis, in accordance with an embodiment.

FIG. 3A illustrates an architecture of the protein backbone transformer of FIG. 2, in accordance with an embodiment.

FIG. 3B illustrates an architecture of components of the protein backbone transformer of FIG. 3A, in accordance with an embodiment.

FIG. 4A illustrates exemplary synthetic protein structures, in accordance with an embodiment.

FIG. 4B illustrates exemplary fold classes, in accordance with an embodiment.

FIG. 5A illustrates inference and/or training logic, according to at least one embodiment.

FIG. 5B illustrates inference and/or training logic, according to at least one embodiment.

FIG. 6 illustrates training and deployment of a neural network, according to at least one embodiment.

FIG. 7 illustrates an example data center system, according to at least one embodiment.

### DETAILED DESCRIPTION

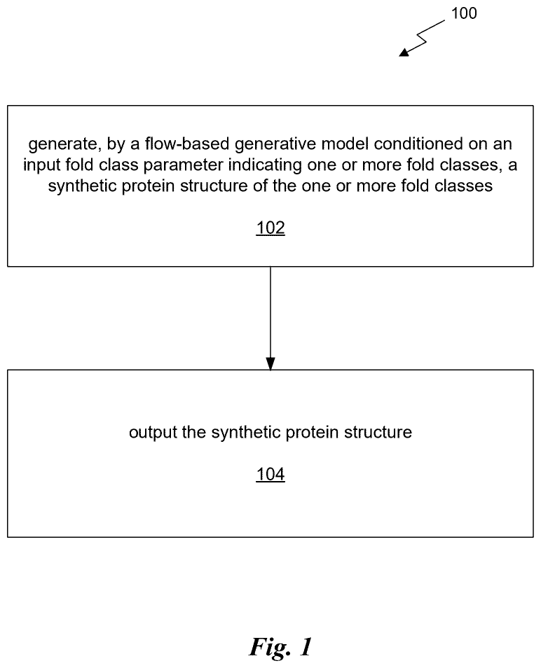

FIG. 1 illustrates a flowchart of a method 100 for protein structure synthesis, in accordance with an embodiment. The method 100 may be performed by a device, which may be comprised of a processing unit, a program, custom circuitry, or a combination thereof, in an embodiment. In another embodiment, a system comprised of a non-transitory memory storage comprising instructions, and one or more processors in communication with the memory, may execute the instructions to perform the method 100. In another embodiment, a non-transitory computer-readable media may store computer instructions which when executed by one or more processors of a device cause the device to perform the method 100.

In operation 102, a synthetic protein structure of one or more fold classes is generated by a flow-based generative model conditioned on an input fold class parameter indicating the one or more fold classes. With respect to the present description, the synthetic protein structure is a computer generated structure that represents a protein. In an embodiment, the synthetic protein structure may be a synthetic protein backbone.

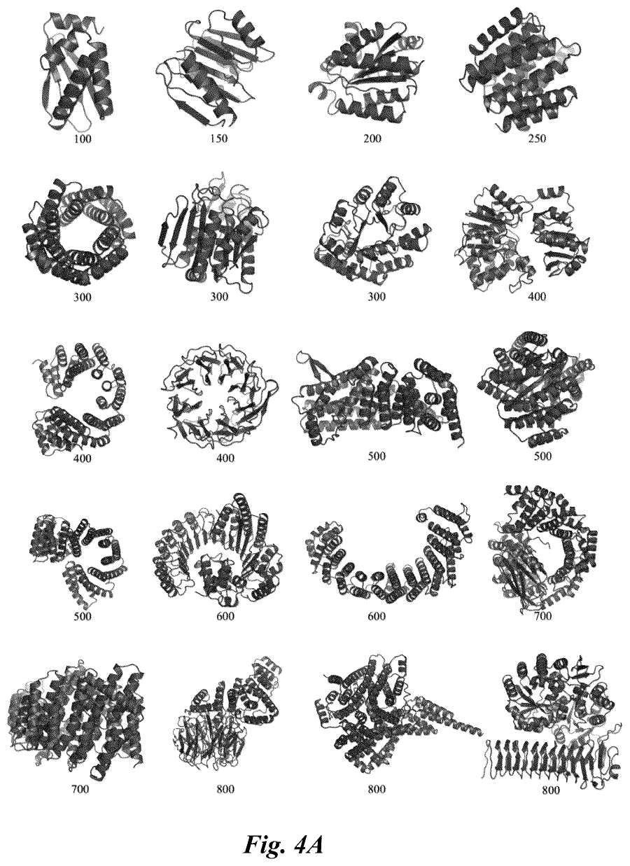

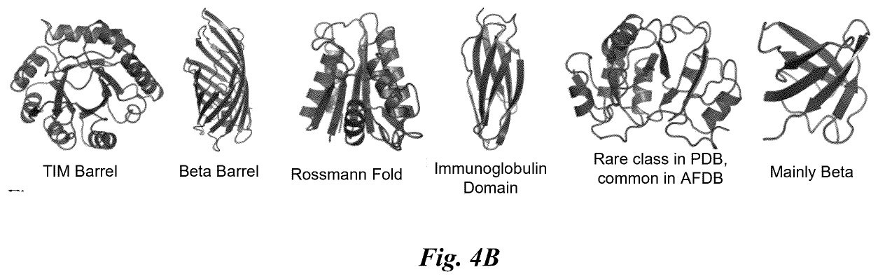

In an embodiment, the synthetic protein structure may be generated such that it is realistic, whereby a physical protein formed (e.g. in a lab) in accordance with the synthetic protein structure can be used in various applications, such as creating medicine for drug development, tissue engineering, regenerative medicine, biotechnology for research and industrial applications, material science for novel materials with unique properties, etc. FIG. 4A illustrates various exemplary (e.g. realistic) synthetic protein structures that may be generated by the flow-based generative model.

As mentioned, the synthetic protein structure is generated by the flow-based generative model to be of one or more fold classes, per an input fold class parameter indicating the one or more fold classes. With respect to the present description, a fold class refers to a predefined category of protein tertiary structure topology. Thus, the input on which the flow-based generative model is conditioned may indicate one or more such predefined categories of protein tertiary structure topology. FIG. 4B illustrates exemplary fold classes.

For example, in an embodiment, the input fold class parameter may indicate a single fold class. In this embodiment, the flow-based generative model may generate a synthetic protein structure of the single fold class. In particular, the flow-based generative model may generate a synthetic protein structure having a tertiary structure topology that conforms with the single fold class.

In another embodiment, the input fold class parameter may be hierarchical. For example, in an embodiment, the input fold class parameter may indicate a primary fold class and a secondary fold class (e.g. an optionally a third fold class and so on). With respect to this embodiment, the input fold class parameter may indicate a first degree to which the synthetic protein structure is to be generated in accordance with the primary fold class and a second degree to which the synthetic protein structure is to be generated in accordance with the secondary fold class, etc. A degree may be denoted by percentage, fraction, text corresponding to a predefined amount (e.g. mostly, few, etc.). In an embodiment, the hierarchical fold class parameter may indicate a class, one or subclasses, one or more further subsclasses, etc. to describe the fold at different levels of granularity. For example, the top-level class may specify the fold only on a coarse level, while subclasses will make the fold more specific, and subsubclasses make the fold even more specific. By using a hierarchical fold class parameter, generation of the synthetic protein structure can be conditioned on folds at different levels of the hierarchy, generating more specific or less specific folds. In this embodiment, the flow-based generative model may generate a synthetic protein structure of multiple fold classes (e.g. having a tertiary structure topology that conforms with the multiple fold classes).

In an embodiment, the flow-based generative model may be trained to generate synthetic protein structures conditioned on one or more specified fold classes. In an embodiment, the flow-based generative model may be a generative model that explicitly models a probability distribution by leveraging normalizing flow. In an embodiment, the flow-based generative model may be trained on training data comprised of sample protein structures labeled with fold class labels. In an embodiment, the fold class labels may be hierarchical to indicate (e.g. a degree of) one or more fold classes for each sample protein structure. In an embodiment, the flow-based generative model may be trained in at least two training stages each using a different set of training data. For example, the at least two training stages may include a first training stage in which the flow-based generative model is trained on a first set of sample protein structures having a sequence length below a defined threshold, and a second training stage in which the flow-based generative model is trained on a second set of sample protein structures having a sequence length above the defined threshold.

In an embodiment, at inference time, the flow-based generative model may generate the synthetic protein structure by creating a sequence representation, creating sequence conditioning features, creating a pair representation, and processing the sequence representation, the sequence conditioning features, and the pair representation by a neural network comprised of multi-head attention layers to generate the synthetic protein structure. In an embodiment, the sequence representation and the pair representation may be created from noisy protein coordinates. In an embodiment, the sequence representation may be created to include registers that capture global information. In an embodiment, the sequence conditioning features may be created from the input fold class parameter.

In an embodiment, the multi-head attention layers may be conditioned on the sequence conditioning features and may be biased through the pair representation to update the sequence representation. In an embodiment, the sequence representation, the sequence conditioning features, and the pair representation may be normalized prior to processing through the multi-head attention layers. In an embodiment, the neural network may be configured to update the pair representation based on the updated sequence representation and to decode the updated pair representation into pairwise distances for the updated sequence representation. In an embodiment, the neural network may be comprised of triangle multiplicative layers for updating the pair representation.

In an embodiment, classifier-free guidance may be used to condition the flow-based generative model on the input fold class parameter. In classifier-free guidance, the flow-based generative model may be guided by an unconditional model. In another embodiment, autoguidance may be used to guide generation of the synthetic protein structure by the flow-based generative model. In autoguidance, the flow-based generative model may be guided by an inferior version of itself (e.g. having less training, capacity, etc.).

In operation 104, the synthetic protein structure is output. In an embodiment, the synthetic protein structure may be output to a computer memory. In an embodiment, the synthetic protein structure may be output for use in generating the physical protein (e.g. in a lab). In an embodiment, the synthetic protein structure and/or the physical protein may be used by a downstream application, as such those applications mentioned above.

To this end, the method 100 may be performed to use a flow-based generative model to generate a synthetic protein structure conditioned on an input specifying one or more fold classes. This conditioning may allow the automated generation of the synthetic protein structure to be controlled such that the resulting synthetic protein structure conforms to the specified fold class(es).

Further embodiments will now be provided in the description of the subsequent figures. It should be noted that the embodiments disclosed herein with reference to the method 100 of FIG. 1 may apply to and/or be used in combination with any of the embodiments of the remaining figures below.

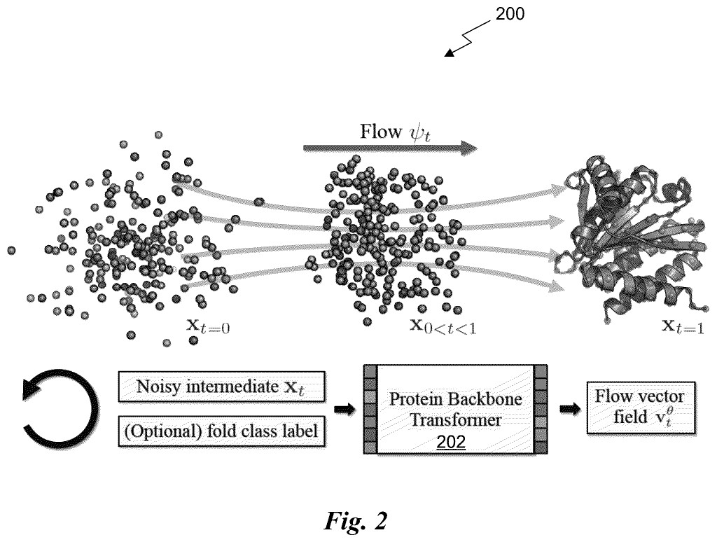

FIG. 2 illustrates a flow-based generative model architecture 200 for protein structure synthesis, in accordance with an embodiment. The flow-based generative model architecture 200 may be implemented to carry out the method 100 of FIG. 1. Thus, the definitions and descriptions provided above may equally apply to the present embodiment.

As shown, the flow-based generative model architecture 200 is comprised of a protein structure transformer 202. The protein structure transformer 202 may be a neural network, in an embodiment. The flow-based generative model architecture 200 relies on flow-matching, which models a probability density path pₜ(x₁) that gradually transforms an analytically tractable noise distribution (pₜ₌₀) into a data distribution (pₜ₌₁), following a time variable t∈[0, 1].

Formally, the path pₜ(xₜ) corresponds to a flow ψₜ that pushes samples from p₀ to pₜ via pₜ=[ψ]ₜ*p₀, where * denotes the push-forward. In practice, the flow is modelled via an ordinary differential equation (ODE)

$${dx}_{t} = {v}_{t}^{θ} ( {x}_{t} , t ) dt ,$$

defined through a learnable vector field

$${v}_{t}^{θ} ( {x}_{t} , t )$$

with parameters θ. Initialized from noise x₀˜p₀(x₀), this ODE simulates the flow and transforms noise into approximate data distribution samples. The probability density path pₜ(xₜ) and the (intractable) ground-truth vector field uₜ(xₜ) are related via the continuity equation

$${dp}_{t} ( {x}_{t} ) / dt = - {∇}_{{x}_{t}} ( {p}_{t} ( {x}_{t} ) {u}_{t} ( {x}_{t} ) ) .$$

To learn

$${v}_{t}^{θ} ( {x}_{t} , t ) ,$$

conditional flow matching (CFM) can be employed. In CFM, conditioned on data samples x₁˜p₁(x₁), conditional probability paths pₜ(xₜ|x₁) are constructed for which the corresponding ground-truth conditional vector field uₜ(xₜ|x₁) is analytically tractable for simple distributions p₀(x₀), such as Gaussian noise. The CFM objective then corresponds to regressing the neural network-defined approximate vector field

$${v}_{t}^{θ} ( {x}_{t} , t )$$

against uₜ(xₜ|x₁), where the intermediate samples xₜ are drawn from the tractable conditional probability path pₜ(xₜ|x₁) and data x₁ is marginalized over via Monte Carlo sampling. Since in expectation the CFM objective results in the same gradients as directly regressing against the intractable marginal ground-truth vector field uₜ(xₜ),

$${v}_{t}^{θ} ( {x}_{t} , t )$$

learns an approximation or the ground-truth uₜ(xₜ).

In practice, the conditional probability paths are defined through an interpolant that connects noise x₀ and data samples x₁ and constructs intermediate xₜ via interpolation. The rectified flow (also known as conditional optimal transport) formulation is relied on, using a linear interpolant xₜ=tx₁+(1−t)x₀ and the regression target dψₜ(x₀|x₁)/dt=x₁−x₀. An embodiment of the CFM objective will be described in more detail below.

### An Embodiment for Scaling Training Data

In an embodiment, the flow-based generative model may be trained on two datasets, denoted as D_{FS} and D_{21M}:

1. Foldseek AFDB clusters D_{FS}: This dataset is based on sequential filtering and clustering of the AFDB with the sequence-based MMseqs2 and the structure-based Foldseek. This data uses cluster representatives only, i.e. only one structure per cluster. Lengths between 32 and 256 residues are used in the main models, leading to 588,571 structures in total.

2. High-quality filtered AFDB subset D_{21M}: All ≈214M AFDB structures are filtered for proteins with maximum residue length 256, minimum average pLDDT of 85, maximum pLDDT standard deviation of 15, maximum coil percentage of 50%, and maximum radius of gyration of 3 nm. This leads to 20,874,485 structures. The data is further clustered with MMseqs2 using a 50% sequence similarity threshold. During training, clusters are sampled uniformly, and random structures within are drawn.

Hierarchical fold class annotations. Existing protein structure diffusion or flow models are either trained unconditionally, or condition only on partially given local structures, for instance in motif scaffolding tasks. In an embodiment, fold class annotations that globally describe protein structures may be leveraged to train the flow-based generative. The Encyclopedia of Domains (TED) data may be used, which consists of structural domain assignments to proteins in the AFDB. TED uses the CATH structural hierarchy to assign labels, where C (“class”) describes the overall secondary-structure content of a domain, A (“architecture”) groups domains with high structural similarity, T (“topology/fold”) further refines the structure groupings, and H (“homologous superfamily”) labels are only shared between domains with evolutionary relationships. In an embodiment, only C, A, and T level labels may be used for the training data. Labels may be assigned to the proteins in all datasets. In an embodiment, the main “mainly α”, “mainly β”, and “mixed α/β” C classes may be used.

### An Embodiment of the Training Objective

Protein backbones' residue locations may be modeled through their C_{α} atom coordinates only. Consider the vector of a protein backbone's 3D C_{α} coordinates x∈R^{3L}, where L is the number of residues. Denote the protein's fold class labels as {Cₓ, Aₓ, Tₓ}_{CAT}, and the binned pairwise distance between residues i and j as D_{b,ij}(x). Using xₜ=tx+(1−t)ϵ, the objective then may be defined per Equation 1.

$$\begin{matrix}min \\ θ\end{matrix} {𝔼}_{x ∼ {p}_{D} ( x ) , ϵ ∼ N ( 0 , I ) , t ∼ p ( t )} [ \frac{1}{L} {\underbrace{{‖ {v}_{t}^{θ} ( {x}_{t} , t , \hat{x} ( {x}_{t} ) , {\{ {C}_{x} , {A}_{x} , {T}_{x} \}}_{CAT} ) - ( x - ϵ ) ‖}_{2}^{2}}}_{main conditional flow - matching loss} - \frac{1 ( t \geq 0.3 )}{{L}^{2}} {\sum }_{i , j} {\sum }_{b = 1}^{64} {\underbrace{{D}_{b , ij} ( x ) log {p}_{b , ij}^{θ} ( {x}_{t} , t , \hat{x} ( {x}_{t} ) , {\{ {C}_{x} , {A}_{x} , {T}_{x} \}}_{CAT} )}}_{optional auxiliary binned distogram loss} ] \qquad Equation 1$$

A cross entropy-based distogram loss may optionally be included, which discretizes pairwise residue distances into 64 bins. The distogram is predicted via a prediction head attached to the architecture's pair representation and only used if this pair representation is updated. This loss is generally used only for t≥0.3. The model may also be trained for self-conditioning, conditioning the model on its own clean data prediction

$$\hat{x} ( {x}_{t} ) = {x}_{t} + ( 1 - t ) {v}_{t}^{θ} ( {x}_{t} , t , ∅ , {\{ {C}_{x} , {A}_{x} , {T}_{x} \}}_{CAT} )$$

with probability 0.5. Furthermore, a novel t-sampling distribution may be used, p(t)=0.02 U(0, 1)+0.98 B(1.9, 1.0), tailored to flow matching for protein backbone generation, where U is a uniform distribution and B a beta distribution.

Fold-class conditioning. The fold class labels describe protein structures at different levels of detail, and the model is trained to both condition on varying levels of the hierarchy, and to also run unconditionally. To this end, different label combinations are hierarchically dropped out during training. Specifically, with p=0.5 all labels ({Ø, Ø, Ø}_{CAT}) are dropped, with p=0.1 only the C label ({Cₓ, Ø, Ø}_{CAT}) is shown, with p=0.15 only the T label ({Cₓ, Aₓ, Ø}_{CAT}) is dropped and with p=0.25 the model is given all labels ({Cₓ, Aₓ, Tₓ}_{CAT}). The drop probabilities are chosen such that, on the one hand, a strong unconditional model is learned without any labels. On the other hand, the number of categories increases along the hierarchy, such that training is focused more on the increasingly fine A and T classes, as opposed to conditioning only on the coarser C labels. This approach enables classifier-free guidance for all possible levels during inference, combining the unconditional model prediction with any of the label-conditioned predictions (guidance weight ω). Note that, while most training proteins have only a single label, if a protein has multiple domains and corresponding hierarchical labels, one of them is randomly fed to the model.

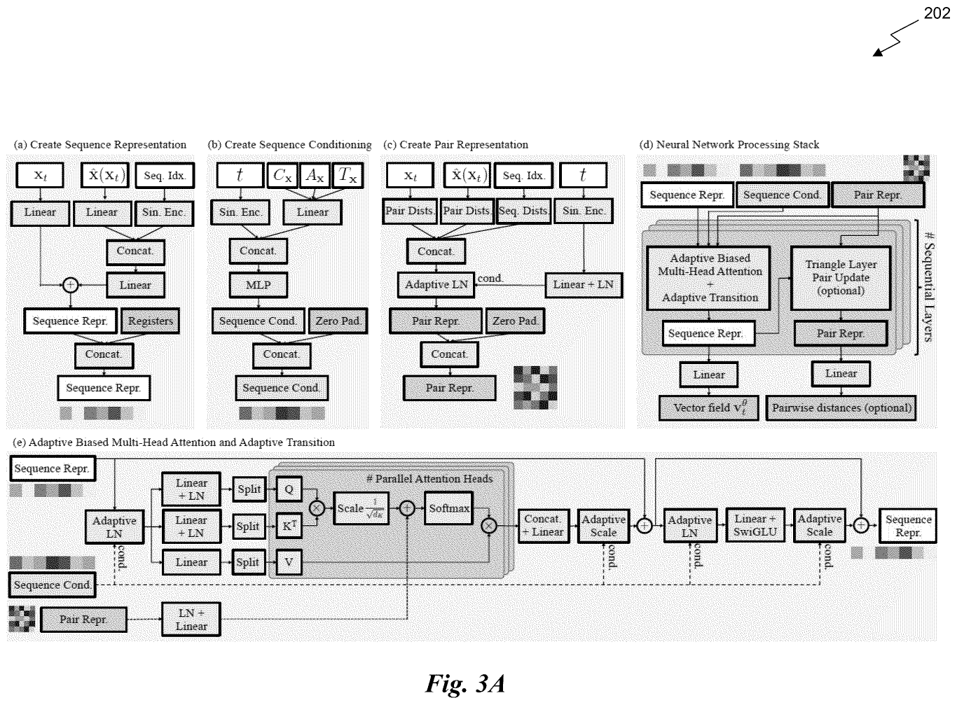

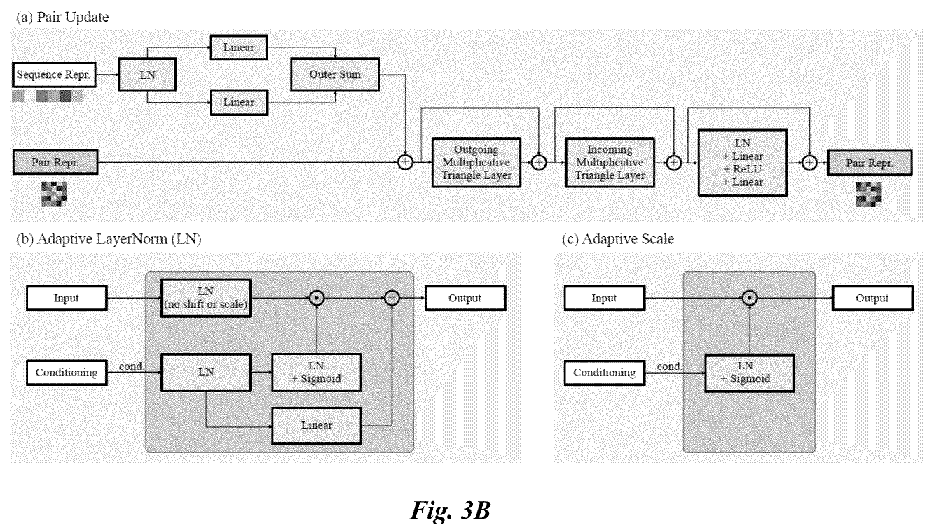

FIG. 3A illustrates an architecture of the protein backbone transformer 202 of FIG. 2, in accordance with an embodiment. In the present embodiment, the protein backbone transformer 202 is a streamlined non-equivariant transformer (e.g. neural network) that constructs residue chain and pair representations from the (noisy) protein coordinates, the residue indices, the sequence separation between residues and the (optional) self-conditioning input. The residue chain representation is processed by a stack of conditioned and biased multi-head self-attention layers, using a pair bias via the pair representation, which can be optionally updated, too. At the end, the updated sequence representation is decoded into the vector field prediction

$${v}_{t}^{θ}$$

to model the flow.

In particular, in (a)-(c) of the transformer 202 shown, a sequence representation, sequence conditioning features, and a pair representation are created. In (d), the sequence representation, sequence conditioning features, and pair representation are processed by conditioned and biased (through the pair representation) multi-head attention layers, described in (e). A variant of QK normalization may be used, applying LayerNorm (LN) to the Q and K inputs to the attention operation, before the multi-head split. Optionally, the pair representation can be updated.

As mentioned, the model is conditioned on hierarchical fold class labels. They are fed to the model through concatenated learnable embeddings, injected into the attention stack via adaptive layer norms, together with the t embedding. The sequence representation is extended with auxiliary tokens, known as registers, which can capture global information or act as attention sinks and streamline the sequence processing. A variant of QK normalization is used to avoid uncontrolled attention logit growth. All of the attention layers feature residual connections to allow for stable training. Triangle multiplicative layers are used as an optional add-on only to update the pair representation.

The model learns the distribution of protein structures without sequence inputs. To learn equivariance, training proteins are centered and augmented with random rotations. In an embodiment, the model may be trained with up to ≈400M parameters in the transformer and ≈17M in the triangle layers.

### Sampling

New protein backbones can be generated using the model by simulating the learnt flow's ODE. Since the flow is Gaussian, there exists a connection between the learnt vector field and the corresponding score s(xₜ):=∇ₓ_{ₜ} log pₜ(xₜ), per Equation 2.

$${s}_{t}^{θ} ( {x}_{t} , \tilde{c} ) = \frac{{tv}_{t}^{θ} ( {x}_{t} , \tilde{c} ) - {x}_{t}}{1 - t} \qquad Equation 2$$

where \tilde{c} is an abbreviation for all conditioning inputs. This allows for a stochastic differential equation (SDE) that can be used as a stochastic alternative to sample the model, per Equation 3.

$${dx}_{t} = {v}_{t}^{θ} ( {x}_{t} , \tilde{c} ) dt + g ( t ) {s}_{t}^{θ} ( {x}_{t} , \tilde{c} ) dt + \sqrt{2 g ( t ) γ {dW}_{t}} \qquad Equation 3$$

where Wₜ is a Wiener process and g(t) scales the additional score and noise terms, which corresponds to Langevin dynamics. A noise scaling parameter γ is also introduced. For γ=1, the SDE has the same marginals and hence samples from the same distribution as the ODE. In an embodiment, noise scale can be reduced in stochastic sampling, for improving designability at the cost of diversity. Fold label conditioning can be done via classifier-free guidance (CFG) or autoguidance. In both approaches, different scores may be combined to obtain a “higher quality” score that leads to improved samples. In a unifying formulation, the guided vector field as can be written per Equation 4.

$${v}_{t}^{θ , guided} ( {x}_{t} , \tilde{c} ) = ω {v}_{t}^{θ} ( {x}_{t} , \tilde{c} ) + ( 1 - ω ) [ ( 1 - α ) {v}_{t}^{θ} ( {x}_{t} , ∅ ) + α {v}_{t}^{θ , bad} ( {x}_{t} , \tilde{c} ) ] \qquad Equation 4$$

where ω≥0 defines the overall guidance weight and α∈[0, 1] interpolates between CFG and autoguidance. An analogous equation holds for the scores

$${s}_{t}^{θ} ( {x}_{t} , \tilde{c} ) .$$

FIG. 3B illustrates an architecture of components of the protein backbone transformer of FIG. 3A, in accordance with an embodiment. In particular, the present embodiment provides an example of the (a) Pair Update, (b) Adaptive LayerNorm (LN), and (c) Adaptive Scale modules of the protein backbone transformer of FIG. 3A.

When creating the pair representation (see FIG. 3A (c)), the pair and sequence distances created from the inputs xₜ, \hat{x}(xₜ) and the sequence indices are discretized and encoded into one-hot encodings. Specifically, for the pair distances from xₜ 64 bins of equal size between 1 Å and 30 Å are used with the first bin being <1 Å and the last one being >30 Å, for the pair distances from \hat{x}(xₜ) 128 bins of equal size between 1 Å and 30 Å are used with the first bin being <1 Å and the last one being >30 Å, and for the sequence separation distances 127 bins may be used for sequence separations [<−63, −63, −62, −61, . . . , 61, 62, 63, >63].

As shown in FIG. 3A, this pair representation can be (optionally) updated throughout the network using pair update layers. These feed the sequence representation through linear layers to update the pair representation, which is additionally updated using triangular multiplicative updates as shown in the present embodiment.

In an embodiment, 10 register tokens may be used when constructing the sequence representation. Sequence conditioning and pair representation may be zero-padded accordingly. The MLP used when creating the sequence conditioning (see FIG. 3A (b)) may correspond to a Linear-SwiGLU-Linear-SwiGLU-Linear architecture.

### Machine Learning

Deep neural networks (DNNs), including deep learning models, developed on processors have been used for diverse use cases, from self-driving cars to faster drug development, from automatic image captioning in online image databases to smart real-time language translation in video chat applications. Deep learning is a technique that models the neural learning process of the human brain, continually learning, continually getting smarter, and delivering more accurate results more quickly over time. A child is initially taught by an adult to correctly identify and classify various shapes, eventually being able to identify shapes without any coaching. Similarly, a deep learning or neural learning system needs to be trained in object recognition and classification for it get smarter and more efficient at identifying basic objects, occluded objects, etc., while also assigning context to objects.

At the simplest level, neurons in the human brain look at various inputs that are received, importance levels are assigned to each of these inputs, and output is passed on to other neurons to act upon. An artificial neuron or perceptron is the most basic model of a neural network. In one example, a perceptron may receive one or more inputs that represent various features of an object that the perceptron is being trained to recognize and classify, and each of these features is assigned a certain weight based on the importance of that feature in defining the shape of an object.

A deep neural network (DNN) model includes multiple layers of many connected nodes (e.g., perceptrons, Boltzmann machines, radial basis functions, convolutional layers, etc.) that can be trained with enormous amounts of input data to quickly solve complex problems with high accuracy. In one example, a first layer of the DNN model breaks down an input image of an automobile into various sections and looks for basic patterns such as lines and angles. The second layer assembles the lines to look for higher level patterns such as wheels, windshields, and mirrors. The next layer identifies the type of vehicle, and the final few layers generate a label for the input image, identifying the model of a specific automobile brand.

Once the DNN is trained, the DNN can be deployed and used to identify and classify objects or patterns in a process known as inference. Examples of inference (the process through which a DNN extracts useful information from a given input) include identifying handwritten numbers on checks deposited into ATM machines, identifying images of friends in photos, delivering movie recommendations to over fifty million users, identifying and classifying different types of automobiles, pedestrians, and road hazards in driverless cars, or translating human speech in real-time.

During training, data flows through the DNN in a forward propagation phase until a prediction is produced that indicates a label corresponding to the input. If the neural network does not correctly label the input, then errors between the correct label and the predicted label are analyzed, and the weights are adjusted for each feature during a backward propagation phase until the DNN correctly labels the input and other inputs in a training dataset. Training complex neural networks requires massive amounts of parallel computing performance, including floating-point multiplications and additions. Inferencing is less compute-intensive than training, being a latency-sensitive process where a trained neural network is applied to new inputs it has not seen before to classify images, translate speech, and generally infer new information.

### Inference and Training Logic

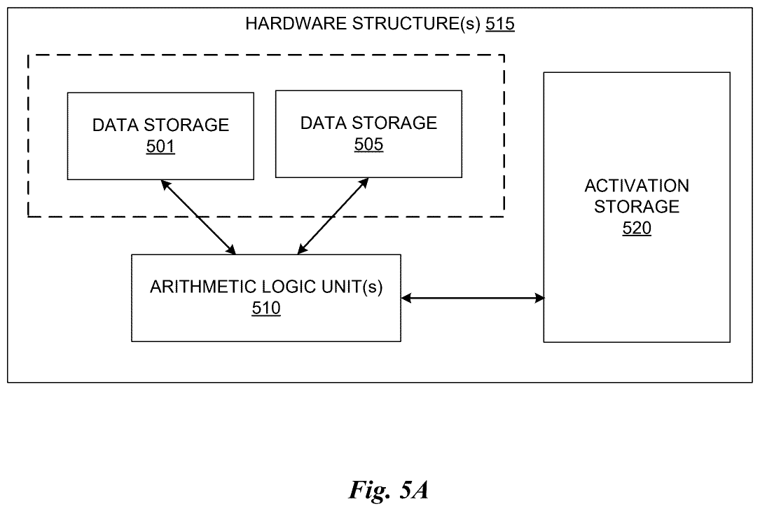

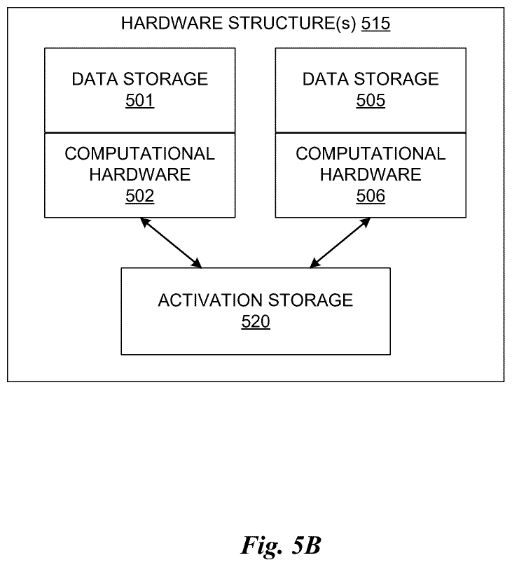

As noted above, a deep learning or neural learning system needs to be trained to generate inferences from input data. Details regarding inference and/or training logic 515 for a deep learning or neural learning system are provided below in conjunction with FIGS. 5A and/or 5B.

In at least one embodiment, inference and/or training logic 515 may include, without limitation, a data storage 501 to store forward and/or output weight and/or input/output data corresponding to neurons or layers of a neural network trained and/or used for inferencing in aspects of one or more embodiments. In at least one embodiment data storage 501 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of data storage 501 may be included with other on-chip or off-chip data storage, including a processor's L1, L2, or L3 cache or system memory.

In at least one embodiment, any portion of data storage 501 may be internal or external to one or more processors or other hardware logic devices or circuits. In at least one embodiment, data storage 501 may be cache memory, dynamic randomly addressable memory (“DRAM”), static randomly addressable memory (“SRAM”), non-volatile memory (e.g., Flash memory), or other storage. In at least one embodiment, choice of whether data storage 501 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 515 may include, without limitation, a data storage 505 to store backward and/or output weight and/or input/output data corresponding to neurons or layers of a neural network trained and/or used for inferencing in aspects of one or more embodiments. In at least one embodiment, data storage 505 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, any portion of data storage 505 may be included with other on-chip or off-chip data storage, including a processor's L1, L2, or L3 cache or system memory. In at least one embodiment, any portion of data storage 505 may be internal or external to on one or more processors or other hardware logic devices or circuits. In at least one embodiment, data storage 505 may be cache memory, DRAM, SRAM, non-volatile memory (e.g., Flash memory), or other storage. In at least one embodiment, choice of whether data storage 505 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors.

In at least one embodiment, data storage 501 and data storage 505 may be separate storage structures. In at least one embodiment, data storage 501 and data storage 505 may be same storage structure. In at least one embodiment, data storage 501 and data storage 505 may be partially same storage structure and partially separate storage structures. In at least one embodiment, any portion of data storage 501 and data storage 505 may be included with other on-chip or off-chip data storage, including a processor's L1, L2, or L3 cache or system memory.

In at least one embodiment, inference and/or training logic 515 may include, without limitation, one or more arithmetic logic unit(s) (“ALU(s)”) 510 to perform logical and/or mathematical operations based, at least in part on, or indicated by, training and/or inference code, result of which may result in activations (e.g., output values from layers or neurons within a neural network) stored in an activation storage 520 that are functions of input/output and/or weight parameter data stored in data storage 501 and/or data storage 505. In at least one embodiment, activations stored in activation storage 520 are generated according to linear algebraic and or matrix-based mathematics performed by ALU(s) 510 in response to performing instructions or other code, wherein weight values stored in data storage 505 and/or data 501 are used as operands along with other values, such as bias values, gradient information, momentum values, or other parameters or hyperparameters, any or all of which may be stored in data storage 505 or data storage 501 or another storage on or off-chip. In at least one embodiment, ALU(s) 510 are included within one or more processors or other hardware logic devices or circuits, whereas in another embodiment, ALU(s) 510 may be external to a processor or other hardware logic device or circuit that uses them (e.g., a co-processor). In at least one embodiment, ALUs 510 may be included within a processor's execution units or otherwise within a bank of ALUs accessible by a processor's execution units either within same processor or distributed between different processors of different types (e.g., central processing units, graphics processing units, fixed function units, etc.). In at least one embodiment, data storage 501, data storage 505, and activation storage 520 may be on same processor or other hardware logic device or circuit, whereas in another embodiment, they may be in different processors or other hardware logic devices or circuits, or some combination of same and different processors or other hardware logic devices or circuits. In at least one embodiment, any portion of activation storage 520 may be included with other on-chip or off-chip data storage, including a processor's L1, L2, or L3 cache or system memory. Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 520 may be cache memory, DRAM, SRAM, non-volatile memory (e.g., Flash memory), or other storage. In at least one embodiment, activation storage 520 may be completely or partially within or external to one or more processors or other logical circuits. In at least one embodiment, choice of whether activation storage 520 is internal or external to a processor, for example, or comprised of DRAM, SRAM, Flash or some other storage type may depend on available storage on-chip versus off-chip, latency requirements of training and/or inferencing functions being performed, batch size of data used in inferencing and/or training of a neural network, or some combination of these factors. In at least one embodiment, inference and/or training logic 515 illustrated in FIG. 5A may be used in conjunction with an application-specific integrated circuit (“ASIC”), such as Tensorflow® Processing Unit from Google, an inference processing unit (IPU) from Graphcore™, or a Nervana® (e.g., “Lake Crest”) processor from Intel Corp. In at least one embodiment, inference and/or training logic 515 illustrated in FIG. 5A may be used in conjunction with central processing unit (“CPU”) hardware, graphics processing unit (“GPU”) hardware or other hardware, such as field programmable gate arrays (“FPGAs”).

FIG. 5B illustrates inference and/or training logic 515, according to at least one embodiment. In at least one embodiment, inference and/or training logic 515 may include, without limitation, hardware logic in which computational resources are dedicated or otherwise exclusively used in conjunction with weight values or other information corresponding to one or more layers of neurons within a neural network. In at least one embodiment, inference and/or training logic 515 illustrated in FIG. 5B may be used in conjunction with an application-specific integrated circuit (ASIC), such as Tensorflow® Processing Unit from Google, an inference processing unit (IPU) from Graphcore™, or a Nervana® (e.g., “Lake Crest”) processor from Intel Corp. In at least one embodiment, inference and/or training logic 515 illustrated in FIG. 5B may be used in conjunction with central processing unit (CPU) hardware, graphics processing unit (GPU) hardware or other hardware, such as field programmable gate arrays (FPGAs). In at least one embodiment, inference and/or training logic 515 includes, without limitation, data storage 501 and data storage 505, which may be used to store weight values and/or other information, including bias values, gradient information, momentum values, and/or other parameter or hyperparameter information. In at least one embodiment illustrated in FIG. 5B, each of data storage 501 and data storage 505 is associated with a dedicated computational resource, such as computational hardware 502 and computational hardware 506, respectively. In at least one embodiment, each of computational hardware 506 comprises one or more ALUs that perform mathematical functions, such as linear algebraic functions, only on information stored in data storage 501 and data storage 505, respectively, result of which is stored in activation storage 520.

In at least one embodiment, each of data storage 501 and 505 and corresponding computational hardware 502 and 506, respectively, correspond to different layers of a neural network, such that resulting activation from one “storage/computational pair 501/502” of data storage 501 and computational hardware 502 is provided as an input to next “storage/computational pair 505/506” of data storage 505 and computational hardware 506, in order to mirror conceptual organization of a neural network. In at least one embodiment, each of storage/computational pairs 501/502 and 505/506 may correspond to more than one neural network layer. In at least one embodiment, additional storage/computation pairs (not shown) subsequent to or in parallel with storage computation pairs 501/502 and 505/506 may be included in inference and/or training logic 515.

### Neural Network Training and Deployment

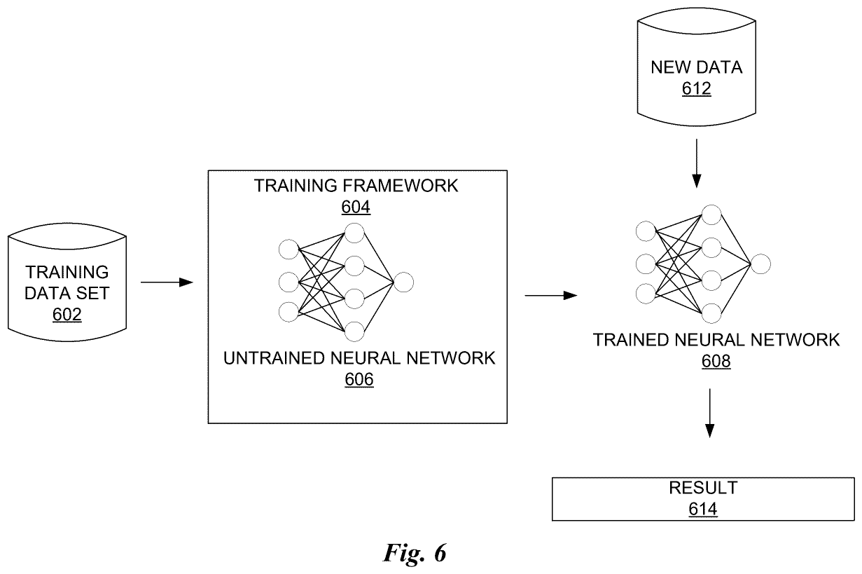

FIG. 6 illustrates another embodiment for training and deployment of a deep neural network. In at least one embodiment, untrained neural network 606 is trained using a training dataset 602. In at least one embodiment, training framework 604 is a PyTorch framework, whereas in other embodiments, training framework 604 is a Tensorflow, Boost, Caffe, Microsoft Cognitive Toolkit/CNTK, MXNet, Chainer, Keras, Deeplearning4j, or other training framework. In at least one embodiment training framework 604 trains an untrained neural network 606 and enables it to be trained using processing resources described herein to generate a trained neural network 608. In at least one embodiment, weights may be chosen randomly or by pre-training using a deep belief network. In at least one embodiment, training may be performed in either a supervised, partially supervised, or unsupervised manner.

In at least one embodiment, untrained neural network 606 is trained using supervised learning, wherein training dataset 602 includes an input paired with a desired output for an input, or where training dataset 602 includes input having known output and the output of the neural network is manually graded. In at least one embodiment, untrained neural network 606 is trained in a supervised manner processes inputs from training dataset 602 and compares resulting outputs against a set of expected or desired outputs. In at least one embodiment, errors are then propagated back through untrained neural network 606. In at least one embodiment, training framework 604 adjusts weights that control untrained neural network 606. In at least one embodiment, training framework 604 includes tools to monitor how well untrained neural network 606 is converging towards a model, such as trained neural network 608, suitable to generating correct answers, such as in result 614, based on known input data, such as new data 612. In at least one embodiment, training framework 604 trains untrained neural network 606 repeatedly while adjust weights to refine an output of untrained neural network 606 using a loss function and adjustment algorithm, such as stochastic gradient descent. In at least one embodiment, training framework 604 trains untrained neural network 606 until untrained neural network 606 achieves a desired accuracy. In at least one embodiment, trained neural network 608 can then be deployed to implement any number of machine learning operations.

In at least one embodiment, untrained neural network 606 is trained using unsupervised learning, wherein untrained neural network 606 attempts to train itself using unlabeled data. In at least one embodiment, unsupervised learning training dataset 602 will include input data without any associated output data or “ground truth” data. In at least one embodiment, untrained neural network 606 can learn groupings within training dataset 602 and can determine how individual inputs are related to untrained dataset 602. In at least one embodiment, unsupervised training can be used to generate a self-organizing map, which is a type of trained neural network 608 capable of performing operations useful in reducing dimensionality of new data 612. In at least one embodiment, unsupervised training can also be used to perform anomaly detection, which allows identification of data points in a new dataset 612 that deviate from normal patterns of new dataset 612.

In at least one embodiment, semi-supervised learning may be used, which is a technique in which in training dataset 602 includes a mix of labeled and unlabeled data. In at least one embodiment, training framework 604 may be used to perform incremental learning, such as through transferred learning techniques. In at least one embodiment, incremental learning enables trained neural network 608 to adapt to new data 612 without forgetting knowledge instilled within network during initial training.

### Data Center

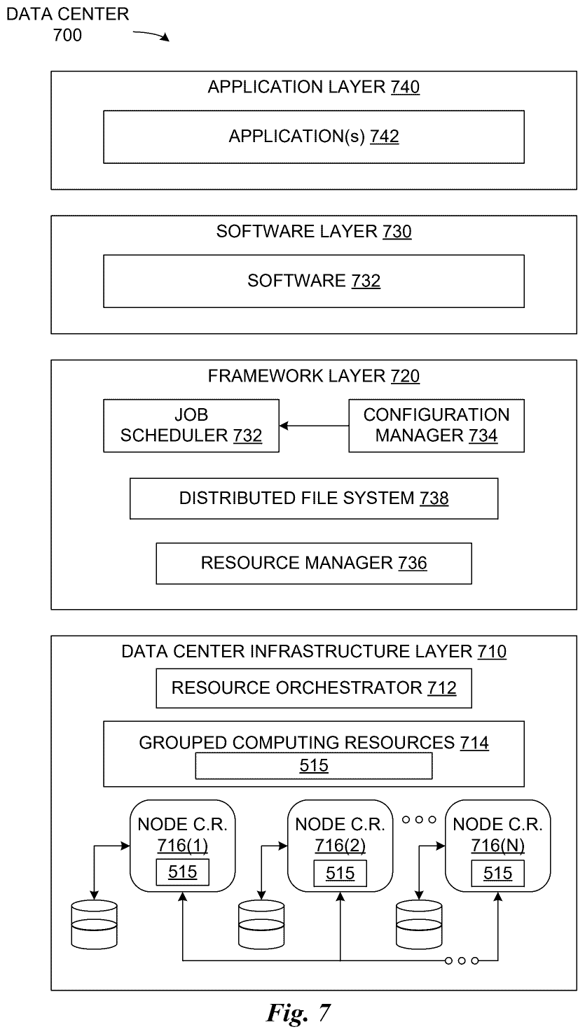

FIG. 7 illustrates an example data center 700, in which at least one embodiment may be used. In at least one embodiment, data center 700 includes a data center infrastructure layer 710, a framework layer 720, a software layer 730 and an application layer 740.

In at least one embodiment, as shown in FIG. 7, data center infrastructure layer 710 may include a resource orchestrator 712, grouped computing resources 714, and node computing resources (“node C.R.s”) 716(1)-716(N), where “N” represents any whole, positive integer. In at least one embodiment, node C.R.s 716(1)-716(N) may include, but are not limited to, any number of central processing units (“CPUs”) or other processors (including accelerators, field programmable gate arrays (FPGAs), graphics processors, etc.), memory devices (e.g., dynamic read-only memory), storage devices (e.g., solid state or disk drives), network input/output (“NW I/O”) devices, network switches, virtual machines (“VMs”), power modules, and cooling modules, etc. In at least one embodiment, one or more node C.R.s from among node C.R.s 716(1)-716(N) may be a server having one or more of above-mentioned computing resources.

In at least one embodiment, grouped computing resources 714 may include separate groupings of node C.R.s housed within one or more racks (not shown), or many racks housed in data centers at various geographical locations (also not shown). Separate groupings of node C.R.s within grouped computing resources 714 may include grouped compute, network, memory or storage resources that may be configured or allocated to support one or more workloads. In at least one embodiment, several node C.R.s including CPUs or processors may grouped within one or more racks to provide compute resources to support one or more workloads. In at least one embodiment, one or more racks may also include any number of power modules, cooling modules, and network switches, in any combination.

In at least one embodiment, resource orchestrator 722 may configure or otherwise control one or more node C.R.s 716(1)-716(N) and/or grouped computing resources 714. In at least one embodiment, resource orchestrator 722 may include a software design infrastructure (“SDI”) management entity for data center 700. In at least one embodiment, resource orchestrator may include hardware, software or some combination thereof.

In at least one embodiment, as shown in FIG. 7, framework layer 720 includes a job scheduler 732, a configuration manager 734, a resource manager 736 and a distributed file system 738. In at least one embodiment, framework layer 720 may include a framework to support software 732 of software layer 730 and/or one or more application(s) 742 of application layer 740. In at least one embodiment, software 732 or application(s) 742 may respectively include web-based service software or applications, such as those provided by Amazon Web Services, Google Cloud and Microsoft Azure. In at least one embodiment, framework layer 720 may be, but is not limited to, a type of free and open-source software web application framework such as Apache Spark™ (hereinafter “Spark”) that may utilize distributed file system 738 for large-scale data processing (e.g., “big data”). In at least one embodiment, job scheduler 732 may include a Spark driver to facilitate scheduling of workloads supported by various layers of data center 700. In at least one embodiment, configuration manager 734 may be capable of configuring different layers such as software layer 730 and framework layer 720 including Spark and distributed file system 738 for supporting large-scale data processing. In at least one embodiment, resource manager 736 may be capable of managing clustered or grouped computing resources mapped to or allocated for support of distributed file system 738 and job scheduler 732. In at least one embodiment, clustered or grouped computing resources may include grouped computing resource 714 at data center infrastructure layer 710. In at least one embodiment, resource manager 736 may coordinate with resource orchestrator 712 to manage these mapped or allocated computing resources.

In at least one embodiment, software 732 included in software layer 730 may include software used by at least portions of node C.R.s 716(1)-716(N), grouped computing resources 714, and/or distributed file system 738 of framework layer 720. one or more types of software may include, but are not limited to, Internet web page search software, e-mail virus scan software, database software, and streaming video content software.

In at least one embodiment, application(s) 742 included in application layer 740 may include one or more types of applications used by at least portions of node C.R.s 716(1)-716(N), grouped computing resources 714, and/or distributed file system 738 of framework layer 720. one or more types of applications may include, but are not limited to, any number of a genomics application, a cognitive compute, and a machine learning application, including training or inferencing software, machine learning framework software (e.g., PyTorch, TensorFlow, Caffe, etc.) or other machine learning applications used in conjunction with one or more embodiments.

In at least one embodiment, any of configuration manager 734, resource manager 736, and resource orchestrator 712 may implement any number and type of self-modifying actions based on any amount and type of data acquired in any technically feasible fashion. In at least one embodiment, self-modifying actions may relieve a data center operator of data center 700 from making possibly bad configuration decisions and possibly avoiding underutilized and/or poor performing portions of a data center.

In at least one embodiment, data center 700 may include tools, services, software or other resources to train one or more machine learning models or predict or infer information using one or more machine learning models according to one or more embodiments described herein. For example, in at least one embodiment, a machine learning model may be trained by calculating weight parameters according to a neural network architecture using software and computing resources described above with respect to data center 700. In at least one embodiment, trained machine learning models corresponding to one or more neural networks may be used to infer or predict information using resources described above with respect to data center 700 by using weight parameters calculated through one or more training techniques described herein.

In at least one embodiment, data center may use CPUs, application-specific integrated circuits (ASICs), GPUs, FPGAs, or other hardware to perform training and/or inferencing using above-described resources. Moreover, one or more software and/or hardware resources described above may be configured as a service to allow users to train or performing inferencing of information, such as image recognition, speech recognition, or other artificial intelligence services.

Inference and/or training logic 515 are used to perform inferencing and/or training operations associated with one or more embodiments. In at least one embodiment, inference and/or training logic 515 may be used in system FIG. 7 for inferencing or predicting operations based, at least in part, on weight parameters calculated using neural network training operations, neural network functions and/or architectures, or neural network use cases described herein.

As described herein, a method, computer readable medium, and system are disclosed to provide protein structure synthesis. In accordance with FIGS. 1-4B, embodiments may provide a flow-based generative model usable for performing inferencing operations and for providing inferenced data (e.g. a protein structure). The flow-based generative model may be stored (partially or wholly) in one or both of data storage 501 and 505 in inference and/or training logic 515 as depicted in FIGS. 5A and 5B. Training and deployment of the flow-based generative model may be performed as depicted in FIG. 6 and described herein. Distribution of the flow-based generative model may be performed using one or more servers in a data center 700 as depicted in FIG. 7 and described herein.

What is claimed is:

1. A method, comprising:
at a device:
generating, by a flow-based generative model conditioned on an input fold class parameter indicating one or more fold classes, a synthetic protein structure of the one or more fold classes; and
outputting the synthetic protein structure.

2. The method of claim 1, wherein the input fold class parameter indicates a single fold class.

3. The method of claim 2, wherein the flow-based generative model generates the synthetic protein structure of the single fold class.

4. The method of claim 1, wherein the input fold class parameter is hierarchical.

5. The method of claim 4, wherein the input fold class parameter indicates a primary fold class and a secondary fold class.

6. The method of claim 5, wherein the input fold class parameter indicates a first degree to which the synthetic protein structure is to be generated in accordance with the primary fold class and a second degree to which the synthetic protein structure is to be generated in accordance with the secondary fold class.

7. The method of claim 1, wherein the synthetic protein structure is a synthetic protein backbone.

8. The method of claim 1, wherein the flow-based generative model generates the synthetic protein structure by:
creating a sequence representation,
creating sequence conditioning features,
creating a pair representation, and
processing the sequence representation, the sequence conditioning features, and the pair representation by a neural network comprised of multi-head attention layers to generate the synthetic protein structure.

9. The method of claim 8, wherein the sequence representation and the pair representation are created from noisy protein coordinates.

10. The method of claim 9, wherein the sequence representation is created to include registers that capture global information.

11. The method of claim 8, wherein the sequence conditioning features are created from the input fold class parameter.

12. The method of claim 8, wherein the multi-head attention layers are conditioned on the sequence conditioning features and are biased through the pair representation to update the sequence representation.

13. The method of claim 8, wherein the sequence representation, the sequence conditioning features, and the pair representation are normalized prior to processing through the multi-head attention layers.

14. The method of claim 12, wherein the neural network is configured to update the pair representation based on the updated sequence representation and to decode the updated pair representation into pairwise distances for the updated sequence representation.

15. The method of claim 14, wherein the neural network is comprised of triangle multiplicative layers for updating the pair representation.

16. The method of claim 1, wherein classifier-free guidance is used to condition the flow-based generative model on the input fold class parameter.

17. The method of claim 1, wherein autoguidance is used to guide generation of the synthetic protein structure by the flow-based generative model.

18. The method of claim 1, wherein the flow-based generative model is trained on training data comprised of sample protein structures labeled with fold class labels.

19. The method of claim 18, wherein the fold class labels are hierarchical to indicate one or more fold classes for each sample protein structure.

20. The method of claim 18, wherein the flow-based generative model is trained in at least two training stages each using a different set of training data.

21. The method of claim 20, wherein the at least two training stages include:
a first training stage in which the flow-based generative model is trained on a first set of sample protein structures having a sequence length below a defined threshold, and
a second training stage in which the flow-based generative model is trained on a second set of sample protein structures having a sequence length above the defined threshold.

22. A system, comprising:
a non-transitory memory storage comprising instructions; and
one or more processors in communication with the memory, wherein the one or more processors execute the instructions to:
generate, by a flow-based generative model conditioned on an input fold class parameter indicating one or more fold classes, a synthetic protein structure of the one or more fold classes; and
output the synthetic protein structure.

23. The system of claim 22, wherein the input fold class parameter indicates a first degree to which the synthetic protein structure is to be generated in accordance with a primary fold class and a second degree to which the synthetic protein structure is to be generated in accordance with a secondary fold class.

24. A non-transitory computer-readable media storing computer instructions which when executed by one or more processors of a device cause the device to:
generate, by a flow-based generative model conditioned on an input fold class parameter indicating one or more fold classes, a synthetic protein structure of the one or more fold classes; and
output the synthetic protein structure.

25. The non-transitory computer-readable media of claim 24, wherein the input fold class parameter indicates a first degree to which the synthetic protein structure is to be generated in accordance with a primary fold class and a second degree to which the synthetic protein structure is to be generated in accordance with a secondary fold class.
